(19) United States
(12) Patent Application Publication
(10) Pub. No.: US 2010/0081184 A1
(43) Pub. Date: Apr. 1, 2010

(54) METHOD FOR EVALUATION, DESIGN AND OPTIMIZATION OF IN-SITU BIOCONVERSION PROCESSES

(76) Inventors: Robert A. Downey, Centennial, CO (US); Marc Ware, Golden, CO (US)

Correspondence Address: CARELLA, BYRNE, CECCHI, OLSTEIN, BRODY & AGNELLO, 5 BECKER FARM ROAD, ROSELAND, NJ, 07068, US

(21) Appl. No.: 12/565,839

(22) Filed: Sep. 24, 2009

Related U.S. Application Data
(60) Provisional application No. 61/100,289, filed on Sep. 26, 2008.

(51) Int. Cl. C12P 5/02; G06F 17/50

(52) U.S. Cl. 435/167; 703/2

(57) ABSTRACT

A method for the evaluation, design and optimization of in-situ bioconversion processes for the conversion of carbon to methane and other useful gases and liquids. The method utilizes a comprehensive computer simulation model for accurately simulating the physical and dynamic conditions in a subterranean carbon-bearing formation and the effects of stimulating the growth of indigenous or non-indigenous microbes therein for the bioconverstion of carbon to methane and other useful gases and liquids. The method enables the prediction of bioconversion rates and efficiencies under a range of variables, and thus provides for the optimization of in-situ bioconversion process design and operation.

This application claims priority on U.S. provisional application Ser. No. 61/100,289 filed Sep. 26, 2008 in the name of Robert Downey et al. incorporated by reference in its entirety herein.

### BACKGROUND OF THE INVENTION

1. Field of the Invention

The present invention relates to a method for the production of methane, carbon dioxide, gaseous and liquid hydrocarbons and other valuable products from subterranean formations, such as coal for example, in-situ, utilizing indigenous and non-indigenous microbial consortia, and in particular, a method for simulating such production and for producing the product based on the simulation.

2. Copending Applications of Interest

Of interest are commonly owned copending patent applications, U.S. application Ser. No. 12/459,416 entitled “Method for Optimizing In-Situ Bioconversion of Carbon Bearing Formations” filed Jul. 1, 2009, U.S. application Ser. No. 12/455,431 entitled “The Stimulation of Biogenic Gas Generation in Deposits of Carbonaceous Material” filed Jun. 2, 2009, both in the name of Robert A. Downey and U.S. application Ser. No. 12/252,919 entitled “Pretreatment of Coal” filed Oct. 16, 2008 in the name of Verkade et al., all incorporated by reference herein.

3. Description of Related Art

According to the United States Geological Survey, the coal-bearing basins of the United States contain deposits of more than 6 Trillion tons of coal. The great majority of these coal deposits cannot be mined due to technical and economic limitations, yet the stored energy in these coal deposits exceeds that of U.S. annual crude oil consumption over a 2000-year period. Economical and environmentally sound recovery and use of some of this stored energy could reduce U.S. reliance on foreign oil and gas, improve the U.S. economy, and provide for improved U.S. national security.

About 8% of U.S. natural gas reserves and production, known as “coalbed methane” are derived from natural gas trapped in some of these coal deposits, and a significant percentage of these gas resources were generated by indigenous syntrophic anaerobic microbes known as methanogenic consortia, that have the ability to convert the carbon in coal, and other carbon-bearing materials, to methane. While these methane deposits were generated over geologic time, if these methanogenic consortia could be enhanced to convert more of the carbon contained in coal, shale or even oil reservoirs to methane gas, the resulting production could significantly add to the natural gas reserves and production.

U.S. Pat. No. 6,543,535, incorporated by reference herein, discloses a process for stimulating microbial activity in a hydrocarbon bearing subterranean formation such as oil or coal. The presence of microbial consortia is determined and a characterization made, preferably genetic, if at least one microorganism of the consortia, at least one being a methanogenic microorganism. The characterization is compared with at least one known characterization derived from a known microorganism having one or more known physiological and ecological characteristics. This information with other information obtained from analysis of the rock and fluid, is used to determine an ecological environment that promotes in situ microbial degradation of formation hydrocarbons and promotes microbial generation of methane by at least one methanogenic microorganism of the consortia and used as a basis for modifying the information environment to produce methane. Thus this process involves the stimulation of preexisting microorganisms to promote methane production.

However, as coal or other hydrocarbon deposits are converted, over time, they diminish in volume and thus reduce the output of the converted deposit. Also the output of such converted deposits are subject to numerous variables that effect the particular output of a given hydrocarbon deposit. Presently, determining the potential output of such deposits is dependent upon the expertise of those of skill in the art to determine the extent of the deposit and from this extent, estimate the potential possible output.

Such estimates are subject however to numerous factors, known or unknown, which may alter the actual output from the estimate. Also such estimates are highly inaccurate, especially for periods of time as the hydrocarbon bed is exhausted, since estimates need also be made as to the rate of exhaustion of such beds over time. Such estimates need to consider a number of variables that may or may not be consistently employed in the estimate. Therefore, the estimated outputs are subject to highly inaccurate factors. Such inaccuracies are undesirable, since implementation of a hydrocarbon deposit conversion process can be costly. This prior process is thus highly inefficient and potentially inaccurate. The present inventor recognizes a need for an improved efficient method to optimize the prediction of methane production from a subterranean hydrocarbon formation. The prior art in this field do not recognize this need nor address it.

### SUMMARY OF THE INVENTION

A method according to one embodiment of the present invention employs a comprehensive mathematical model that describes the geological, geophysical, hydrodynamic, microbiological, chemical, biochemical, geochemical, thermodynamic and operational characteristics of systems and processes for the in-situ bioconversion of carbon-bearing subterranean formations to methane, carbon dioxide and other hydrocarbons using indigenous or non-indigenous methanogenic consortia, via the introduction of microbial nutrients, methanogenic consortia, chemicals and electrical energy, and the operation of the systems and processes via surface and subsurface facilities.

A method according to a second embodiment of the present invention is for the design, implementation and optimization of systems and processes for the in-situ bioconversion of carbon-bearing subterranean formations to methane, carbon dioxide and other hydrocarbons using indigenous or non-indigenous methanogenic consortia via the introduction of microbial nutrients, methanogenic consortia, chemicals and electrical energy, utilizing a comprehensive mathematical model that fully describes the geological, geophysical, hydrodynamic, microbiological, chemical, biochemical, geochemical, thermodynamic and operational characteristics of such systems and processes.

The method according to a further embodiment includes utilizing the model for assessing the extent and location of the bioconversion of materials in the subterranean deposit formation to methane, carbon dioxide and/or other hydrocarbons.

The method according to a further embodiment includes manipulating, adjusting, changing or altering and controlling the bioconversion of materials in the subterranean formation to methane, carbon dioxide and of the bioconversion process via comparing actual operational results and the data to model-predicted results.

The method according to a further embodiment includes determining or estimating the volumes and mass of subterranean formation, porosity, fluid, gas, nutrient and biological material at any given time before, during and after applying the method according to the one and second embodiments.

The method according to a further embodiment includes determining the amount of carbon in the subterranean formation that is bioconverted to methane, carbon dioxide and other hydrocarbons, at any given time before, during and after applying the method according to the one and second embodiments.

A process for producing a gaseous product by bioconversion of a subterranean carbonaceous deposit according to a third embodiment comprises bioconverting a subterranean carbonaceous deposit to the gaseous product by use of a methanogenic consortia, said bioconverting being operated based on a mathematical simulation that predicts production of the gaseous product by use of at least (i) one or more physical properties of the deposit; (ii) one or more changes in one or more physical properties of the deposit as result of said bioconverting; (iii) one or more operating conditions of the process; and (iv) one or more properties of the methanogenic consortia.

The process according to a still further embodiment wherein the one or more physical properties of the deposit comprise depth, thickness, pressure, temperature, porosity, permeability, density, composition, types of fluids and volumes present, hardness, compressibility, nutrients, presence, amount and type of methanogenic consortia.

The process according to a further embodiment where the operating conditions comprise one or more of injecting into the deposit: a predetermined amount of the methogenic consortia, a predetermined amount of water at a predetermined flow rate, and a predetermined amount of a given nutrient.

The process according to a further embodiment wherein the properties of the methanogenic consortia include the types and amount of consortia.

The process according to a further embodiment wherein the gaseous product is one of methane and carbon dioxide.

The process according to a further embodiment wherein the gaseous product is at least one gas, the process including recovering the at least one gas from the deposit.

The process according to a further embodiment wherein the process includes recovering the at least one gas from the deposit and the simulation includes dividing the deposit in to at least one grid of a plurality of three dimensional deposit subunits, and predicting the amount of recovery of the at least one gas from one or more subunits.

The process according to a still further embodiment wherein the simulation includes dividing the deposit into a grid of a plurality of three dimensional subunits, selecting the subunit exhibiting an optimum amount of gaseous product to be recovered and then recovering the bioconverted product from that selected subunit.

The process according to a further embodiment including recovering the gaseous product from the deposit wherein the simulation includes dividing the deposit into at least one grid of a plurality of three dimensional deposit sectors, and predicting the amount of recovery of the at least one gas from one or more sectors, and determining the flow of the gaseous product from sector to adjacent sector.

The process according to a further embodiment wherein the simulation comprises the steps of FIGS. 2a and 2b.

### BRIEF DESCRIPTION OF THE DRAWING

FIG. 1 is a representative schematic plan view of a subterranean deposit of a hydrocarbon bed useful in explaining certain principles of the present invention;

FIG. 1a is an isometric view of a portion of the deposit and related terrain of FIG. 1; and

FIGS. 2a and 2b is a flow chart showing the steps of a prediction model for the determination of an optimized desired fluid output for a given hydrocarbon subterranean bed.

### DETAILED DESCRIPTION OF THE PREFERRED EMBODIMENTS

Microbial methanogenic consortia, either indigenous or non-indigenous to the carbon-bearing subterranean formation of interest, such as coal for example, are capable of metabolizing carbon and converting it to desired and useful components such as methane, carbon dioxide and other hydrocarbons. The amount of these bioconversion component products that are produced, and the rate of such production, is recognized in the present embodiment as a function of several factors, including but not necessarily limited to, the specific microbial consortia present, the nature or type of the carbon-bearing formation, the temperature and pressure of the formation, the presence and geochemistry of the water within the formation, the availability and quantity of nutrients required by the microbial consortia to survive and grow, the presence or saturation of methane and other bioconversion products or components, and several other factors. Therefore the efficient bioconversion of the carbon-bearing subterraneous formation to methane, carbon dioxide and other hydrocarbons require optimized methods and processes for the delivery and dispersal of nutrients into the formation, the dispersal of microbial consortia across the surface area of the formation, the exposure of as much surface area of the formation to the microbial consortia, and the removal and recovery of the generated methane, carbon dioxide and other hydrocarbons from the formation.

The rate of carbon bioconversion is proportionate to the amount of surface area available to the microbes utilized in the conversion process, the population of the microbes and the movement of nutrients into the deposits and bioconversion products extracted from the deposit as the deposit is depleted. The amount of surface area available to the microbes is proportionate to the percentage of void space, or porosity, of the subterranean formation; and the permeability, or measure of the ability of gases and fluids to flow through the subterranean formation is in turn proportionate to its porosity. All subterranean formations are to some extent compressible, i.e., their volume, porosity, and permeability is a function of the net stress upon them. Their compressibility is in turn a function of the materials, i.e., minerals, hydrocarbon chemicals and fluids, the porosity of the rock and the structure of the materials, i.e., crystalline or non-crystalline. It is believed that by reducing the net effective stress upon a carbon-bearing subterranean formation, the permeability, porosity, internal and fracture surface area available for bioconversion can be improved and thus the ability to move nutrients, microbes and generated methane, carbon dioxide and other hydrocarbons into and out of the subterranean deposit formation. Most coals and some carbon-bearing shale formations have much greater compressibilities than other strata, such as sandstones, siltstones, limestones and shales. Coals are the most compressible of all carbon-bearing rock types, and thus their net effective stress, porosity and permeability may be most affected by alterations in formation pressure.

Subterranean carbon-bearing formations may at any time be saturated with fluids, such as liquids and/or gases, and such saturations also affect the net effective stress on the formations. The permeability of gases and liquids in the subterranean formation is also dependent upon their saturations, and thus by purposefully increasing the pressure within the subterranean formation well above its initial condition, to an optimum point, and maintaining that pressure continuously, it is believed that the flow of fluids, nutrients, microbial consortia and generated methane, carbon dioxide and hydrocarbons may be optimized. The optimum pressure point of the process may be determined initially by utilization of mathematical relationships that define permeability of the subterranean formation as a function of net effective stress, such as the correlation presented by Somerton et al. (1975):

$$k = {k}_{0} [ exp ( \frac{0.003 Δ σ}{{( {k}_{0} )}^{0.1}} ) + 0.0002 {( Δ σ )}^{1 / 3} {( {k}_{0} )}^{1 / 3} ]$$

Where:

K₀=original permeability at zero net stress, millidarcies

K=permeability at new stress Δσ

Δσ=net stress, psia

The maximum pressure in which the process may be reasonably operated may be limited by that point at which the fluid pressure in the subterranean formation exceeds its tensile strength, causing fractures to form and propagate in the formation, in either a vertical or horizontal plane, as determined by Poisson's ratio. These pressure-induced fractures may form large fluid channels through which the injected fluids nutrients and microbial consortia and generated methane may flow, thus reducing or inhibiting distribution of fluid pressure and reduction of net effective stress throughout the subterranean formation.

Operation of the conversion process at a subterranean formation at a pressure point above initial or hydrostatic conditions and at optimum net effective stress will enable better determination of inter-well permeability trends and changes in inter-well permeability as the process proceeds. The bioconversion of solid coal or shale to methane gas reduces the solid volume of the coal or shale along the surfaces, and thus will increase the fracture aperture and pore diameter of the relevant porosities. The increases in fracture aperture and pore diameter will increase the permeability of the subterranean formation, and the efficiency of the conversion process.

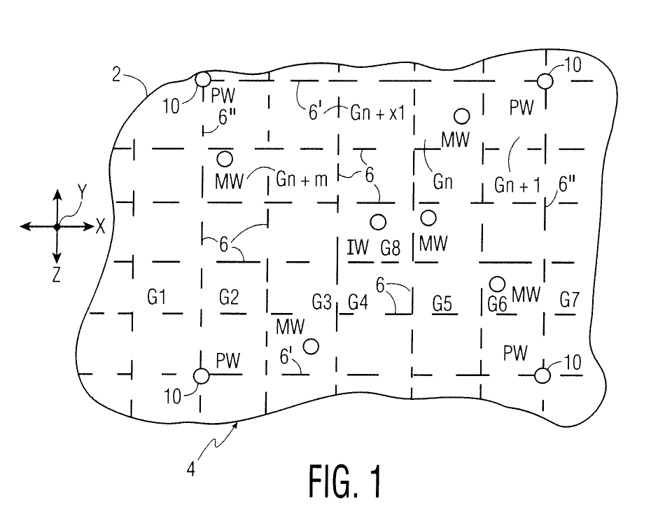

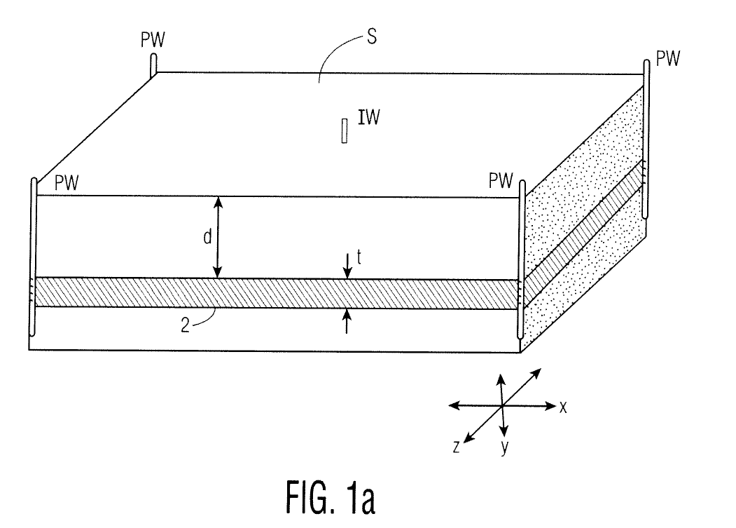

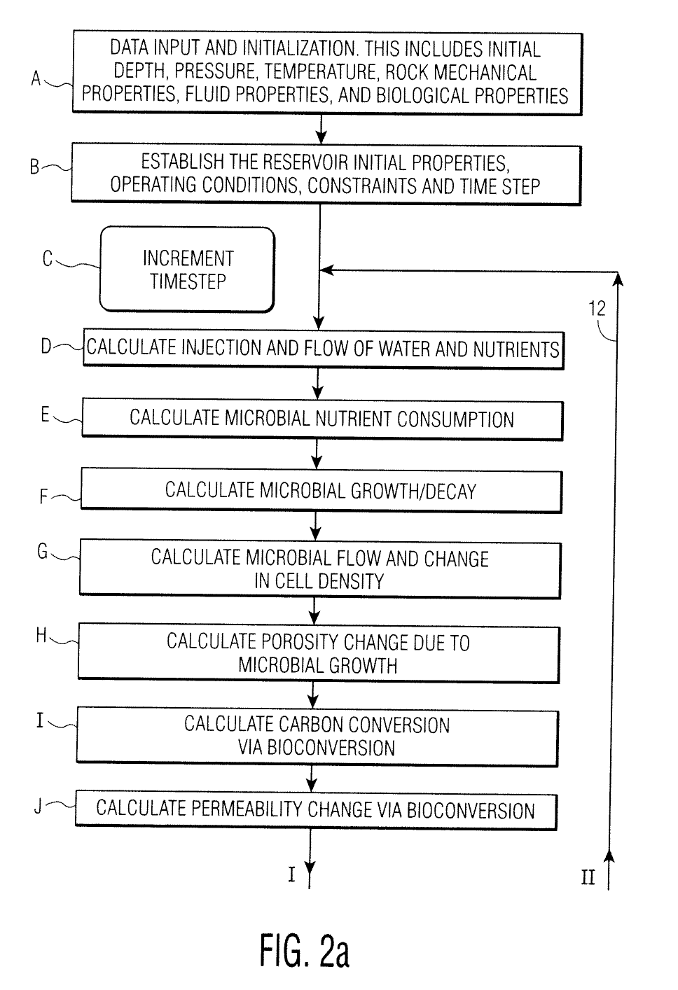

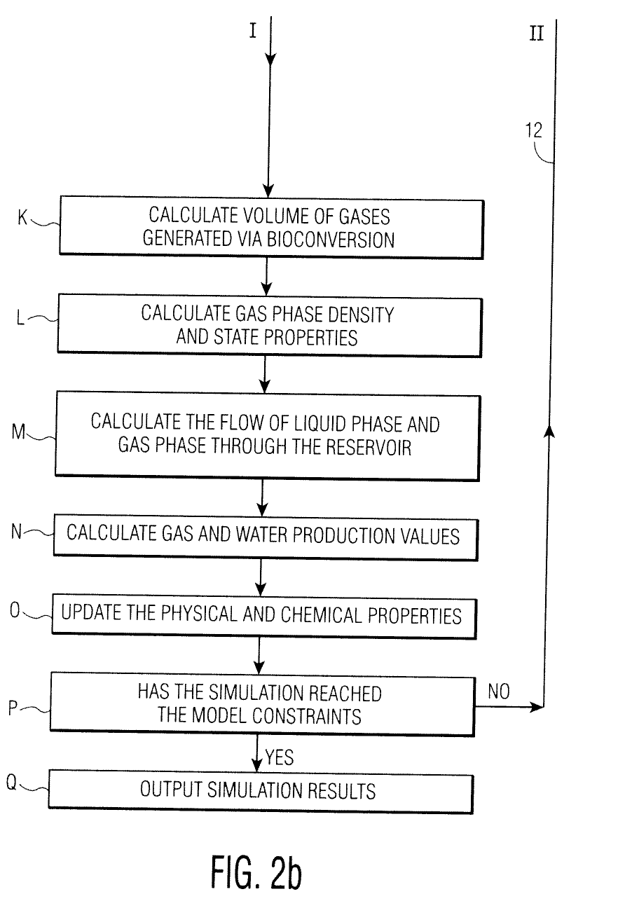

Many carbon-bearing subterranean formations have multiple types of porosity, or pore space, a function of the type of material it is comprised of and the forces that have been and are exerted upon it. Many coal seams, for example, have dual or triple porosity systems, whereby pore spaces may exist as fractures, large matrix spaces and/or small matrix spaces. These pore spaces may vary substantially across an area, may exhibit directional trends or orientations, and also may be variable in the vertical orientation within the subterranean formation. The permeability of subterranean formations may also vary substantially a really and vertically within a given subterranean environment. Given sufficient geological and geophysical data, a number of characteristics of a subterranean formation such as thickness, areal extent, depth, slope (not shown in the figures), (See FIGS. 1 and 1a) saturation, permeability, porosity, temperature, formation geochemistry, formation composition, and pressure may be ascertained and a 3-dimensional mathematical model of the subterranean formation and these characteristics may be developed. Such a model is presented by the equations discussed below and which implements the process of FIGS. 2a and 2b, to be discussed below.

The mathematical model in one non-limiting embodiment herein may be constructed so as to provide for subdivision of the subterranean formation into relatively small three dimensional polygon or sectors of the foundation such as cubes or rectangles, FIGS. 1 and 1a, the assumed locations of points where inputs into and out of the subterranean formation may be made, and a range of characteristic conditions may be applied at any location or upon any of the polygons, as a function of time. These polygons and so on are each assigned unique identifications G1-n. The polygons are formed as an array which is assigned a value in the corresponding computer program in which the unique assigned IDs are also entered. The entire array of grids is thus entered into the relevant computer program, which can then access each grid individually for that deposit. In FIG. 1, for example, the grids are assigned unique IDs G1, G2, G3, G4, G5 and so on to Gn for all of the grids created for this terrain.

In FIG. 1a, a subterranean formation 2 of hydrocarbon, for example coal, has a thickness t which in practice, is variable and not a constant value as illustrated by way of simplicity of illustration in this exemplary figure. In FIG. 1, the geographical extent of the formation 2 in terrain 4 may have any peripheral dimension in the x, z (horizontal) and y (vertical) directions and may be in terms of miles (Km) for example. In FIG. 1, the terrain 4 is divided into three dimensional identically dimensioned sectors or grids G1 and so on over the reservoir of the hydrocarbon deposit shown by broken lines 6, which grids G1-n may be cubic (as shown) or rectangular grid blocks (not shown). The grids G1-n are shown in a Cartesian coordinate system x, z (horizontal) and y (vertical). However, this is for purposes of illustration. The grids, in an alternative embodiment, may be divided by radial lines emanating from a common point (not shown) and circumferential lines intersecting the radial lines to define three dimensional frusto-conical blocks with circular segment concentric boundaries (not shown) or into any other grid system. This grid system is incorporated into a computer program that implements the prediction process discussed below as represented by FIGS. 2a and 2b. In FIGS. 2a and 2b, the letters I and II show continuations of the steps from one figure to the other.

In practice, a geologist maps the coal seam deposit formation 2 in the illustrative embodiment using geological mapping software (not shown) that is publicly available. The mapping includes the area extent (width and length), the thickness of the deposit formation and the variation of such thickness over the geographical extent mapped, whether the seam is inclined and where and how much and so completely describes the physical layout of the deposit. This information is translated into the pre-identified grids described above into the geological computer program so a calculation model computer program (FIGS. 2a and 2b) then can be created which identifies all of the physical properties discussed above associated with each grid. The geological program also knows the extent of each grid horizontally (x-z directions) and vertically (y directions). The parameters of the corresponding deposit in each grid is assumed the same and is based on a sample deposit core measured in a laboratory and taken from one or more of the grids.

A non-limiting mathematical calculation model per FIGS. 2a and 2b as discussed below enables the iterative prediction of a plurality of responses in terms of generation of a particular desirable component such as methane of the subterranean formation deposit in response to a range of assumed inputs, such as the injection of fluids, i.e., gases or liquids, such as water and so on, into the subterranean formation in a given assigned grid G1-n and the production of the desired output fluids, liquids and/or gases from the subterranean formation, such as methane, for example. Other models may be constructed in accordance with the invention based on the teachings herein and, therefore, the present invention is not limited to the following model and equations for providing a model.

Laboratory measured physical properties of the subterranean formation, e.g., coal, is determined from a core sample and other data taken at an injection well, such as injection well IW, FIGS. 1 and 1a. These properties include the mechanical properties of the deposit such as Young's modulus of Elasticity, rock compressibility, the measured formation characteristics with regard to its porosity and permeability, microbial content, water volume present and so on, which determination of properties is determined as known in this art.

One or more mathematical calculation prediction models, as disclosed herein below, predicts the effect of a plurality of different values of the injection and withdrawal of different materials such as water, microbes, nutrients, other fluids and/or gases, such as methane, for example, on various parameters of the deposit. These parameters may include pressure, permeability, microbes, nutrients, porosity and fluid movement within and throughout at various locations as defined by the grids G1-n across the subterranean formation based on the laboratory measured initial core values.

These predictions are made over a wide variety of assumed changes in anticipated parameters including time steps, and materials that are inputted into an injection well IW, FIGS. 1 and 1a, including assumed values in iterative simultaneous equations calculations based on the equations given below. These anticipated parameters are based on the measured core and other data obtained from the injection well IW and possibly measured data at other wells such as production wells PW and monitoring wells PM and as measured in a laboratory to ascertain inputs at the injection well IW(s).

Certain of the wells are for monitoring the effect at different points in the formation during a production process. The monitoring determines the effect of the predictions and may result in the altering of the values of the assumed inputs into the injection well(s) to accommodate changes in inputs.

The predicting calculation process according to an embodiment of the present invention includes inputting the description of the deposit as to at least one or more of its: geological, hydrodynamic, microbiological, chemical, biochemical, geochemical, thermodynamic and operational characteristics using indigenous or non-indigenous methanogenic consortia (microbes) via the introduction of microbial nutrients, methanogenic consortia, chemicals, and electrical energy. This will be explained more fully below.

In the well bores of FIGS. 1 and 1a, injection well IW, monitoring wells MW and production wells PW are shown by way of example. In practice there may be many more such wells. These bores are conventional per se in construction, above and below the terrain surface, and can be oriented vertically, horizontally or inclined relative to gravity. The injection bore at well IW is where a core sample of the deposit is taken and measurements of initial data are made of the hydrocarbon deposit 2. Measurements are made at this well which measurements include the depth d of the deposit from the surface S (FIG. 1a), the porosity of the deposit 2, the pressure, the temperature, the microbial activity, mechanical properties of the deposit, and all related measured parameters of the deposit. The core is examined in a laboratory to determine all of such properties initially.

An injection well IW is one in which fluids such as water, microbes, nutrients and/or other materials are injected the amounts of which are assumed based on common knowledge previously known in this art as having a known effect on the deposit based on known equations. The input of materials that are injected into the deposit in assumed amounts may be determined by the laboratory evaluation of the core and then based on such measurements assumptions are made as to the amount of materials to be injected.

The calculation prediction model of the described equations and the process of FIGS. 2a and 2b then utilizes this initial assumed data and inputs to perform the calculations, the initial assumed data may be then modified according to the prediction calculation model results. This initial data taking step from the deposit 2 is illustrated in step A, FIG. 2a. The initial data is, for purpose of illustration rather than limitation, as to the number of wells utilized. At this well bore, the initial reservoir properties, operating conditions, constraints and time step are established based on the measured data and empirically determined.

These properties establish initial conditions including constraints and parameters comprising, for example, measured pressure, the temperature of the reservoir, density of the core sample, weight per unit volume, porosity, Young's Modulus, cleat spacing and so on and included with all of the measured variables taken from the deposit core at the IW site as required by the below described calculation model equations. These measured parameters as well as the assumed inputted injected material parameters such as amount of microbes, the amount of water, and the amount of nutrients that are injected and so on, are inputted into a computer program which performs the calculations in the calculation model.

The calculations of the calculation model are based on simultaneous equation solutions of each of certain of the equations using identical parameters for all equations employing that parameter. The applicable parameter is assigned a tolerance for purpose of providing the same parameter values for all of the equations employing that parameter. That is, a parameter variable appearing in more than one equation is determined by a calculated solution of simultaneous equations so that the parameter value so determined is within the predetermined assigned tolerance.

A tolerance for a computed parameter may be, for example 0.001, 0.0001 and so on, of the value of each relevant parameter in the equation (s) that is being determined by the calculations. For example, if more than one equation uses a given parameter variable, such as ø or p and so on, then the same variable value that falls within that predetermined tolerance is computed as applicable and inserted by the computer program into each equation requiring that variable. The calculations computed for all of the equations is sequential for the process of FIGS. 2a and 2b, but in repetitive occurring loops as shown, until a result is reached for each parameter within its predetermined tolerance. The tolerances may be the same or different for the various different variables and are determined empirically.

The calculations thus performed produce iterative output predictions of the amount of recovery of at least one microbial converted component, e.g., methane, from the deposit. In the equations below, the gas to be recovered is referred to as a gas g. The predictions created by the calculations are utilized for optimizing the recovery from the deposit of the at least one desired converted component of the hydrocarbon deposit, such as methane or others, for example. To produce such a calculation computer program for the calculations performed on such equations is within the skill of those of ordinary skill in the related arts.

The prediction calculation model predicts the effects of the introduction of microbes and other materials such as nutrients for the microbes on the microbes. For example, these effects include microbe predicted growth and the predicted effect of the microbes on the deposit. The amount of microbes being carried by fluids flowing within the subterranean formation are based on predicted characteristics of the formation according to the laboratory measured characteristics inputted into the mathematical calculation model. The model includes a calculation of the generation of a prediction of the microbial attaching to the surfaces of the deposit, a prediction of the microbial growth in population by cell division in the presence of assumed introduced nutrients, a prediction in microbial reduction in population by cell death, and a prediction in the microbial utilization introduced nutrients as an injected fluid.

The prediction includes, for example, a prediction of the effects of the introduction of nutrients, i.e., microbial activity for example, a prediction of how the nutrients may move throughout the formation, a prediction of the consumption of the nutrients by the microbes, a prediction of the metabolic products of the nutrients such as volatile fatty acids, acetate, methane and carbon dioxide produced, a prediction of the absorption or desorption of these metabolic products within the subterranean formation, a prediction of the flow of the metabolic products within the subterranean formation, a prediction of the metabolic products produced from the subterranean formation and removed to the ambient atmosphere surface above the formation, a prediction of the utilization of the microbes for the generation and production of methane, carbon dioxide and other hydrocarbons components from the formation. These predictions are made for each grid G1-n in the terrain 4.

An optimum recovery of the desired component may be ascertained from all of the calculations for all of the grids G1-n. That grid G exhibiting an optimum output as compared to the other grids is selected for placement of a production gas recovery well.

With such predictions, as described below, an optimum component recovery prediction is determined from a plurality of predictions based on different assumed input parameters including the determined data from the core sample. Such different input data is determined, for example, utilizing the predetermined laboratory analysis of the core sample. The optimum component recovery prediction is taken from all of the generated predictions and is selected corresponding to the optimum recovery at a production well(s) of the desired component(s) such as methane and so on for one or more grids exhibiting a corresponding production recovery value. Once the optimum prediction(s) is selected, based on a plurality of predictions based on the different assumed inputted parameters from such materials as water, nutrients, and microbes, then the inputs as determined as described including assumed parameter inputs corresponding to that selected prediction, are implemented in a production mode at the injection well(s) IW to initiate the recovery of the component(s).

The desired component is then recovered at the production well PW, FIGS. 1 and 1a, in the selected grid G1-n or wells (in the specified grids) according to a given implementation. Periodically, core samples are again taken at the IW or at other locations as deemed feasible for a given deposit, and the prediction process repeated and compared to the prior process results to determine if the amounts and types of inputted materials into the injection well need to be reset or reestablished. The production wells then are utilized to recover the desired component on the basis of the new inputs and new prediction(s). This process is repeated as often as might be deemed necessary for a given deposit using assumed values as needed based on general knowledge available to those of ordinary skill in this art.

With an understanding of the constituents, spatial distribution and other characteristics of the subterranean formation as initially measured, and an understanding of the effect of the microbes interacting with the subterranean formation in the biological conversion formation carbon-bearing matter to methane, carbon dioxide and other hydrocarbon products, the mathematical calculation prediction model comprising the equations set forth below is implemented in the process of FIGS. 2a and 2b. This model is utilized to predict the changes in the subterranean formation as a result of the conversion of the deposit to the desired component due to its consumption by the microbes. Such changes may include vertical and areal in terms of volume, porosity, permeability, microbial factors and composition under a range of conditions.

The bioconversion of the carbon-bearing subterranean formation proceeds, solid matter is converted to gases and liquids, such as methane, carbon dioxide, and volatile fatty acids, as well as other hydrocarbons and solids fines. This reduces the volume of the solid matter. This reduction in the solid volume of the carbon-bearing subterranean formation deposit substantially changes the composition of the remaining solid material, as well as changes the porosity and permeability of the subterranean deposit formation. Also changed is the deposit's spatial distribution of porosity and permeability, and the volume of fluids, microbes, and nutrients and their flow, distribution and concentration within the subterranean formation. Such changes are introduced into the calculations using the equations of the prediction calculation model for making further predictions using the exemplary process of FIGS. 2a and 2b.

In FIG. 2a, in step A, the data discussed above is inputted and the system initialized via the computer program that implements the equations described below. The initial data is inputted into the program, the data being taken from the geological survey of the deposit, and also from the extracted core taken from the deposit at the exemplary IW including depth, pressure, temperature, mechanical properties of the deposit material removed core such as density, porosity, permeability, Young's modulus of elasticity, cleat spacing, and so on and fluid properties including salinity, density of the extracted water sample, compressibility of the extracted water sample, which is a function of its salinity.

With respect to the grids G, the grids are tracked by the model in the identified array of grids forming the deposit. This array, comprises the entire deposit structure, is stored in a matrix of grids, each grid with a unique ID in the calculation program. The location of each grid in the array is noted and entered into the program and corresponds to its assigned ID. The size of each grid is entered into the program. The values of the parameters entered at step A are assumed the same for and are entered for each grid.

The calculations are processed for every grid in the system, using calculated input parameter values for each grid as explained below. For example, there may be a number of different values of input parameters utilized in a given grid G1-n based on parameter computations of the next adjacent prior computed grid whose calculated output serves as input data for the next to be computed grid. The program holds these values and utilizes such values for each successive computation for each grid G1-n in the calculation. The laboratory tests and evaluations determine the ideal amounts of the measured data and empirically assumed determined values are inserted for all other values not measured from the core sample at step A.

The inserted data also includes the biological properties such as the number of cells, i.e., microbes (methanogenic consortia) per ml. of fluid, how fast they grow, i.e., how fast they divide, how long they live as the cells decay or cell loss, how fast they are capable of converting carbon into methane and so on. The mechanical and biological properties include all such properties including those noted above and those that are well known to those of ordinary skill in this art. The microbes attach themselves to the core material or float freely in the water extracted with the core sample. Certain of these properties are inputted into the equations discussed below. Thus all of the conditions involved need to be described initially.

These conditions include the geological survey data, i.e., the size and orientation and related properties of the deposit, the assumed size of the grids dividing the surveyed terrain, and the assumed number of wells and location in the array of grids including injection wells IW. The production recovery wells PW may be determined after the calculations are made. This determination is based on the results which determine which grid(s) exhibit optimum recovery in respect of the possible production recovery based on the calculations for all grids G1-n.

Experiments may be run in the laboratory initially to determine ideal amounts of inputted materials which amounts are adjusted initially during such experiments to determine possible methane generation based on the assumed and measured data. The best of such data may then be utilized as the inputs for the calculations of the process of FIGS. 2a and 2b.

Then based on the information obtained as described in the aforementioned paragraphs, an assumption is made as to the likelihood of a certain maximum recovery of at least one desired component whether it be methane, carbon dioxide or any other component material based on the amount of hydrocarbons in the deposit. This recovery, if estimated for a gas such as methane, would estimate the recovery in volume of gas produced such as m³/hour or /day or other unit of time. The estimate would include the total time that at that estimated rate of production, the hydrocarbon would be converted to the desired component, for example, 10, 20 or 30 years and so on, and the deposit exhausted. Such production recovery estimates are within the skill of those skilled in this art and is believed to be commonly made manually in inefficient ways presently on newly discovered deposits.

Once the estimate of the desired production is made, either empirically and/or by laboratory experiment, then data is inputted representing the variables needed for such an estimated production recovery and estimated time period, utilizing the estimated volume of injected water, the volume or amount of microbes, the amount or volume of nutrients required, the pressure in the deposit and so on.

A time step is established, i.e., assumed and entered, at step B, FIG. 2a, for the inputs at step B. These inputs include pressure in the well, the flow of water into the well, the temperature of the water being injected, the amount of nutrients that are being injected with the water, the composition of the nutrients, and so on all of which are preselected at step B based on the initial estimate and also for subsequent various iterations involved in the prediction process for calculating and achieving the desired production recovery. In step B, the reservoir (the deposit or formation) initial properties are established for the reservoir (the deposit), operating conditions, constraints and time step.

The initial properties include the grid data, FIG. 1, the size of the terrain 4, the size of the grids G1-n, the thicknesses of the grids G1-n, angles of the deposit and so on. The grids are located in the Cartesian coordinates x, z in the horizontal directions and y in the vertical direction. The entered data includes the number of wells, injection IW, monitoring MW and producing wells. PW, FIG. 1a, and their locations in the grid. This data includes the properties of the geological formation of the deposit. These properties are well known as to how to measure by known software by those of skill in this art. This data is exported from the geologist's software (or manually if desired) into the process of FIG. 2a at steps A and B, and the equations set forth below are processed by a further computer program which implements these equations.

Conditions are established at which the various wells will be operated at based on the initial estimates. By way of example, at an injection well IW, assume an injection rate of fluids at the rate of a maximum of N number of barrels of liquids per day (24 hrs) maximum and a minimum of N−a barrels per day and the injection will be at a maximum of b psi and a minimum of X−c psi, (the values N, X, a, b and c here used and in the following paragraphs are not related to the equations depicted below) which values can not be exceeded and serve as limits on the production recovery. These values are entered into the computer program model as constraints.

The producing well PW may have a condition of pumping solvents or gases, and it is estimated, for example, that it will produce a maximum of 200 barrels per day of liquids or X m³ of gas(s) per day or a minimum of N-a barrels a day. Constraints or limits are established for this estimate. The constraints include the operating conditions placed on the injection well(s) IW including the maximum production desired for a production well made in the initial estimate for the measured deposit and corresponding to a given time period that the well is operated at.

Another constraint is the time step. A time step is the time required for each calculation of the prediction which is conducted over a period of time (a week, a month, a year etc.) in increments determined by the time step value. The calculations in the prediction process each occur over various assumed time periods entered into the program as a constraint based on an initial estimate of time. These time periods may be different than that required to convert and exhaust the deposit. Initially the time step tells the calculation model the maximum no. of steps, e.g., 10-100,00, as to how long to run the simulation of the process of FIGS. 2a and 2b, e.g., a week, a year, 10 years, 30 years and so on.

Successive time steps of a given value are utilized to provide a maximum conversion prediction of the deposit. Adjustments are made in the time step depending upon the results obtained. For example, using a time step of 0.1 days over a period of 30 days will take about one week of computing time to do all of the calculations utilizing all time steps. In the event no change in result occurs, then the time step is adjusted and the calculations repeated. The process does not care as to the number of time steps utilized in a given predicted time period, e.g., 20 years and so on.

Eventually equilibrium is reached (an equilibrium result is where the calculation reaches a point where all identical parameters in the equations below have identical values within its preset tolerance), or the specified constraints are reached without a result (the simultaneous equation solution for the certain involved equations can not be determined), then the program stops. If a calculation equilibrium results, i.e., each unknown parameter of all of the equations are determined with its corresponding tolerance, regardless of the number of loops of calculations involved between steps P and C, FIGS. 2a and 2b, then the amount of generated gas, i.e., methane, is provided by the equations.

Another constraint is the range of recovery values of the desired component at the production well(s) as originally estimated. These assumed values are inputted and calculations made in the iterative process occurring over the inputted time step periods and the results compared for all grids.

For example, assume a central injection well IW, FIGS. 1 and 1a, and four producing wells PW. Assume that there is an injection rate of 200 barrels of water per day plus nutrients of a further certain amount over a period of 0.1 days. The model, steps D-O, FIGS. 2a, 2b, for that time step performs that calculation for a given assumed period and will assume that that amount of water mass goes into the grids closest to the injection well and will calculate the effect of that occurring over that time step on all other grids in the calculation employing all of the equations below, per steps D-O.

In the various steps, the calculation is made using various equations as follows. Step D, equations 1, 3 and 4, in step E, equation 4 is used, step F, equation 3 is used, in steps G, H and I, equation 2 is used, in step J, equation 6 is used, in step K, equation 5 is used, in step L, equation 5 is used, and in step M, equations 7 and 8 are used.

The flow is computed in the X direction only for one set of calculations using all of the equations of the process, FIGS. 2a and 2b, for all grids. Then the process will go to the next time step at step C, FIG. 2a, and repeat the calculations iteratively for all time steps until an equilibrium output is reached or if not reached, a new set of input data provided until an equilibrium result is provided. Another set of calculations may be made for the Z or Y directions and the process repeated accordingly for all grids.

The changes that occur in a time step determines if new data is to be entered. If no changes in any of the parameters occur in any of the time steps, then new input data is selected and the calculations begun anew. It is expected as the deposit is converted there, will be noticeable changes in the deposit. If not, then the process as computed is not acceptable and restarted with new data and new time steps.

The equations below calculate a mass balance. The calculation model process calculates the effect in the deposit both biologically and from a physical mass stand point across each of the grids G in the deposit sequentially. The model (the equations below), steps D-O, calculates those nutrients in each grid G1-n, and which come in contact with the corresponding microbes, which microbes grew a certain amount in the relevant time period, the microbes had a certain amount of cell division, and consumed a certain amount of nutrients in that time period, and also converted a corresponding amount of the deposit, coal for example. The calculation model repeats the calculation for each grid G1-n, FIG. 1, based on outputs from a prior grid who output flows into that next grid and then at step P determines if the simulation has reached the model operating condition within the constraints set initially at step B, FIG. 2a.

This means that the calculation for identical parameters in the various equations for each grid is the same during the calculation for that grid, but may have different absolute values in the different grids based on a flow of materials as calculated from a prior grid whose output flows to that next succeeding grid, and the equilibrium point for the calculations is reached based on the entered constraints or limits within the tolerance limits as preset for each parameter that is determined in the calculations.

The operating constraints relate to the fact that as the process continues, gas is produced and recovered. For example, as the gas saturation in the deposit increases, the microbes at the same time are producing this gas by converting the deposit, and the gas so produced will flow, and also flow, saturated in, with the water to the producing gas recovery wells. As a result, there is an increased production of gas and less water flowing in the various grids. If the initial constraints do not produce more than the exemplary 200 barrels of liquid a day, a point will be reached where there is more gas being produced than water. In this case the producing wells will not be able to meet the initial constraint liquid flow range in the time step and/or production rate.

Thus certain of the constraints set the limits for such production of fluids per unit time step and thus account for the changes in the deposit. In this case, because there is more gas and less water, the constraint of the minimum amount of water will not be met at the production well, then at step P the process reverts to steps B and C. The constraints, and the time step, are changed at steps B and C as manifested by the arrow 12, FIGS. 2a, 2b, and the process repeated. If the well can not produce the estimated 200 barrels a day, because there is so much gas extracted, then the constraints are changed accordingly and a new production prediction is generated for at least the one desired component, e.g., methane, at a production product recovery well PW.

Another constraint is the setting of a certain tolerance level in reaching a solution to the process of FIGS. 2a and 2b, step P, as discussed. In this process, the variables are reiterated via arrow 12 from step P if the process has not reached the constraint(s) limits or equilibrium with respect to the values of the identical parameters in each of the equations employing that parameter. The process makes certain assumptions about the change and values in the variables, and recalculates in the interactive process where it is trying to reach a value X=value Y for the corresponding variables. Thus the process reiterates over and over again from step P (decision=no) to step C until it reaches a condition wherein a limiting condition is met, step P (decision=yes) where the result is reached that all variables of a given set of equations using that variable, have the same variable value within the tolerance range and the equations reach a solution. This decision indicates that the result is sufficiently close to the desired result and the solution reached is the final solution.

For example, if the process determines that the value of a given variable is within 0.0001 of X=X₂ it is satisfied that the calculation is complete for this variable and ready for inputting the next time step, providing all variables have met this condition. When all time steps are completed, then the process at step Q outputs the results. The number and period of time steps is determined empirically based on the initial terrain and deposit geometries and measured parameters as would be understood by those of ordinary skill.

The tolerance is made sufficiently small so that the process eventually will terminate, otherwise it will keep running. Whenever the value of a parameter of the equations being determined does not change by more than the tolerance value, equilibrium is reached for that variable, and the process repeated for all variables. In this case, when all variables have reached equilibrium, the desired output conditions have been met on each grid in a given sequence in the calculation of the equations. However, these output conditions may or may not match the desired end result estimated production outputs. In this case new estimated data is entered and the process repeated.

The process of FIGS. 2a and 2b calculates the mass flow across each grid G1-n in the X direction from one side of the grid to the other or to the middle of the grid according to a given implementation. So in each time step, a calculation is made for each grid G1-n of the mass flow in direction X.

By way of example, the injection that is made at grid G8 and grid G₁₀₀ (not shown) is examined. At the end of a first time step of 0.1 day, the pressure is 101 psi. The model says this is too high. Something needs to be changed. So the time step is changed. The pressure eventually is 100 psi, then the model says this is acceptable. When all corresponding parameters in all of the equations of the model agree, then the process is completed. If the time step is too large, it is reduced and recalculation is made until the result is within the desired tolerance. Change may occur in all of the grids each time a change is made in the process.

The various characteristics of the formation and the fluids, including the microbes and nutrients therein will vary with changes in pressure, temperature, saturation and flow of such fluids to and among the grids among other parameters as a function of the conversion process.

In step D the injection and flow of water and nutrients is made using equations 1, 3 and 4. Equation 1 provides the flow of water. What the equation is saying is that whenever there is a deformable force media as in coal for example, a change in porosity occurs as a result of the deformation or dissolution of the deposit. The ground water flow follows the equation contingent upon that change in porosity or based on the value of that porosity. The inverted triangle represents the flow of water injected into the injection well IW.

As microbes are added, the porosity will change and so does the amount of flow of water. The last minus term in equation 1 is the change in porosity in relation to the change in time. Eventually this equation will equate to zero. If the last term is made positive, it will be positioned on the other side of the = sign on the right. This means as water is pumped into the deposit, the porosity is changing per unit of time because of the dissolution of the deposit by the microbes, which is the first term on the left of the equation. As the porosity of the rock changes due to microbial activity, this affects the flow rate in the deposit. Thus the injection of water in the injection well IW is utilized by equations 1, 3 and 4. This results in a change in number of microbes and a growth in the decay rate of the microbes.

All of the equations of the calculation model are known in the art. What is unique is their combination and utilization in the process of FIGS. 2a and 2b.

Equation 5 predicts the amount of methane or other gas that will be produced. The amount of gas is represented by the term C_{g} in the equation. The term C_{g} is computed.

Equations 7 and 8 relate to what happens to the gas in the system from time step to time step, i.e., determining the flow. They describe the amount of gas in the water in the system from grid to grid. This provides information how the gas flows in the desired X direction through the system in the same direction from grid to grid. The gas leaves one grid and enters the next grid and so on. Gas that may flow vertically in the Y direction may still flow in the X direction. X and Y are independent of each other however. The equations are concerned with a two dimensional flow X, Y.

In a three dimensional system, flow in the transverse Z direction is recomputed as if in the X direction and the process repeated as described for the X direction. That is the process of the calculation model is run twice, once for the X direction and once for the Z direction. The velocity in the Y direction will not effect these computations.

In each time step, the position of each grid is reinserted. Within each grid there is only so much gas generated in the X and Z directions for a given set of inputs. Thus there are two outputs for the X, Z directions as contemplated by the present process.

Steps E-M are self evident from FIGS. 2a and 2b taken in conjunction with the corresponding equations noted above. The variables are defined in the paragraph after the equations and in Table 1.

The sequence of computation of the equations does not matter in the calculation of equation 5.

In equation 6, permeability does not affect the amount of gas formed. It is a measure of the flow of fluids through the deposit. The position of this calculation in the sequence thus is arbitrary and could be at any position in the diagram of FIGS. 2a and 2b.

The below illustrated mathematical model implemented in the process of FIGS. 2a and 2b is constructed for predicting the production outputs in view of the introduction of various elements or materials as discussed above into the injection well IW, FIGS. 1, 1a and 2a, 2b, according to one embodiment of the prediction model. The various inputs into the equations are based on laboratory measurements of the core and determine the various factors related to the determination of the estimated output desired at the production well(s) PW. These gas or other component recovery outputs are determined iteratively and repeated until the optimum recovery output (the initial estimate of what is desired for this deposit) is reached.

When this occurs, the corresponding estimated materials are inputted at the injection well IW by well known apparatus (not shown) that correspond to the determined calculated optimum production recovery output as iteratively determined by the following calculation model process. At this time, the production wells are utilized to extract and recover the desired fluids and materials by well known apparatus (not shown) at a selected grid based on the calculated output for that grid in comparison to all other grids. The product component recovery extraction process is continued for the time period established by the model. The outputs are monitored at the monitoring wells based on the original data entered into the model corresponding to the selected production mode.

One of ordinary skill by examining the prediction calculation model below can readily determine the parameters to be inputted that are determined in a laboratory based on the core sample taken from the deposit at a well IW and those empirically determined values that need be assumed based on geological data for the deposit and known information in the field about such inputs. For example, the concentration of nutrients is an input value, the change in concentration of the nutrients is measured in a lab, the velocity of water is an estimated input, and so on. Certain of these are assumed empirically and others determined in a laboratory.

The location of such wells may be determined empirically, and/or by periodic use of the calculation model with new inputs or by measurements taken at strategically located wells in the various grids G based on actual production occurring in real time on a periodic basis depending upon the values determined at each well. One of ordinary skill would look at the list of variables and the definitions of the variables and would be able to tell which one are laboratory data, which need to be assumed empirically and so on. The equations calculate how much product, e.g., gas, i.e., methane, water and so on are generated at each grid G1-n. Thus, the calculations for each grid will provide the flow to each grid of gas and water from a previous grid and thus the amount of such fluids can be determined for each production recovery well. The monitoring wells confirm the prediction and manifest the production recovery progress as compared to the prediction.

Step O updates the physical and chemical properties. This resets the initial conditions set in steps A and B. The properties need to be updated after each time step and if no changes occur during calculations. All the properties in each grid block need to be reset accordingly. If the pressure is changed by a change in porosity, the nutrient concentration may also have changed the microbial concentration after a time step. Then a new time step is commenced. Eventually the model reaches the conditions at which the model is shut down and the calculations cease.

The model could be run for example for prediction of a 30 year period or until there is no deposit left or some other condition at which the process is stopped. This reveals how much gas, e.g., methane, or other desired material, is recovered from a production well(s). When step P is reached, the model is asking if it is finished. The model is run until equilibrium, as discussed above, is reached. If equilibrium is reached in two time steps, then the time step value is changed accordingly. The period is set to obtain the assumed desired amount of production recovery. If that amount does not result from a given time period, or the constraints stop the calculations, then the time periods or constraints are reset. A factor is how many iterations the model makes to reach equilibrium, based on tolerance levels and preset constraints.

For example, a condition is imposed for an m time period and injects m1 amount of water and m2 amount of nutrients and so on. (the term m is not used in the equations, but only for this explanation) Then everything is recalculated across the grids of the terrain. If equilibrium does not occur, within the tolerance defined, for each parameter of the equations for each grid, then the time period is changed, e.g., shortened, using a smaller increment of time step, until the within tolerance value for each variable of the equations is reached. There needs to be a balance achieved for all variables. That is, the flow of water from grid to grid should correspond. There is a check and balance in the process.

If certain amount of nutrients are consumed based on laboratory measurements, and microbial amounts decrease, there should be a certain amount of desirable gas produced, recovered, and accounted for. If there is no correlation between consumption and what is produced and recovered, something is wrong. That is, for every amount of nutrient consumed, and change in porosity or other parameter of the deposit, there should be a certain amount of the at least one component, e.g., gas produced, and so on of desired product.

### The Mathematical Calculation Prediction Calculation Model

Equation 1:

This describes dissolution of coal by microbial activities in a deformable porous media:

$$[ {α}_{s} ( 1 - φ ) + {α}_{w} φ ] \frac{\partial p}{\partial t} + ∇ \cdot {q}_{w} - \frac{\partial φ}{\partial t} = 0$$

The term q_{w} refers to flow of water. The addition of microbes changes the porosity of the formation due to consumption by the microbes and thus indicates the effect of the microbes on the consumption of the deposit.

Equation 2:

This describes how porosity changes as a function of microbial cell concentration as a function of the breakdown of the deposit due to microbial consumption (i.e., the conversion via bioconversion from step I, FIG. 2a.

$$\frac{\partial φ}{\partial t} = \frac{{k}_{hyd}}{{ρ}_{coal}} {c}_{bac} φ$$

Equation 3:

Describes the total concentration of microbes increases due to growth or may decrease due to death. This equation describes microbial growth and decay as a function of nutrient supply and mortality rate. This accounts for the increase of microbial density in the system due to consumed nutrients and bioconversion.

$$\frac{\partial {c}_{bac} φ}{\partial t} + ∇ \cdot ( φ {u}_{w} {c}_{bac} - φ D \cdot ∇ {c}_{bac} ) = {μ}_{max} \frac{{c}_{bac} {c}_{nut}}{{K}_{s} + {c}_{nut}} φ - {k}_{d} {c}_{bac} φ$$

Equation 4:

Describes nutrient consumption by microbes:

$$\frac{\partial {c}_{nut} φ}{\partial t} ∇ \cdot ( φ {u}_{w} {c}_{nut} - φ D \cdot ∇ {c}_{nut} ) = - {Y}_{nut / bac} {μ}_{max} \frac{{c}_{bac} {c}_{nut}}{{K}_{s} + {c}_{nut}} φ$$

Equation 5:

Describes the concentration of gas as a function of microbial growth and nutrient consumption:

$$\frac{\partial {c}_{g , w} φ}{\partial t} ∇ \cdot ( φ {u}_{w} {c}_{g , w} - φ D \cdot ∇ {c}_{g , w} ) = {Y}_{g / bac} {μ}_{max} \frac{{c}_{bac} {c}_{nut}}{{K}_{s} + {c}_{nut}} φ$$

Equation 6:

Permeability is expressed by:

$${k}_{xx} = {k}_{yy} = \frac{{{d}_{p}^{2} ( 1 - φ )}^{3}}{150 {( 1 - φ )}^{2}}$$

Equation 7:

Darcy's velocity is:

$${q}_{x} = - \frac{{k}_{xx}}{{μ}_{w}} \frac{\partial p}{\partial x} ;$$

$${q}_{y} = - \frac{{k}_{yy}}{{μ}_{w}} \frac{\partial p}{\partial y}$$

Equation 8:

Velocity of gas phase is expressed by:

$${u}_{gx} = \frac{{u}_{wx}}{φ} ;$$

$${u}_{gy} = \frac{{u}_{wy}}{φ} + {u}_{b}$$

Variable Definition
aₛ Compressibility of coal matrix
a_{w} Compressibility of water
ø porosity
k_{hyd} Hydrolysis coefficient for coal
p Water pressure
q_{w} Darcy velocity
c_{bac} Concentration of microbes
P_{coal} Density of coal
μₘₐₓ Maximum specific growth reaction rate
cₙᵤₜ Concentration of nutrients
c_{g} Concentration of gas
K_{S} Half saturation constant for nutrient
k_{d} Microbe death rate
Y_{nut/bac} Yield coefficient for consumption of nutrient
Y_{g/bac} Yield coefficient for production of gas
T Temperature
P_{g} Density of gas
P_{w} Density of water
u_{w} Velocity of water
u_{g} Velocity of gas
The subscripts xx, yy represent both phase and x (horizontal) or y (vertical) direction. gx=gas in the x direction, wy=water in the y direction, gy=gas in the y direction.
G represents the force of gravity.
The inverted triangle represents a gradient, which is a vector field which points in the direction of the greatest rate of increase of the scalar field.
D Hydrodynamic dispersion coefficient

The units of the above variables and constants are given below in Table 1.

TABLE 1

| Measurement | English Units | Metric Units |
|---|---|---|
| Compressibility of coal matrix | 1/psia | 1/(Pa) |
| Compressibility of water | 1/psia | 1/(Pa) |
| Porosity | ft³/ft³ | m³/m³ |
| Hydrolysis coefficient for coal | Hr⁻¹ | s⁻¹ |
| Water pressure | Psia | Pa |
| Darcy velocity | m/s | m/s |
| Concentration of microbes | pound/ft³ | kg/m³ |
| Density of coal | Pound/ft³ | kg/m³ |
| Maximum specific growth | 1/s | 1/s |
| reaction rate | | |
| Concentration of nutrients | pound/ft³ | kg/m³ |
| Concentration of gas | pound/ft³ | kg/m³ |
| Half saturation constant for | Pound/ft³ | kg/m³ |
| nutrient | | |
| Microbes death rate | 1/s | 1/s |
| Yield coefficient for | Pound of Microbes/ | Kg of Microbes/ |
| consumption of nutrient | pound of nutrients | kg of nutrients |
| Yield coefficient for production | kg of gas/kg of | kg of Gas/kg of |
| of gas | microbes | microbes |
| Temperature | F. | C. |
| Density of gas | Pound/ft³ | kg/m³ |
| Density of water | Pound/ft³ | kg/m³ |
| Hydrodynamic dispersion | in²/minute | m²/s |
| coefficient | | |

All of the above equations are known in this art. What is new is the use of such equations and other equations for developing a mathematical solution that can be used in a process for bioconverting a subterranean cargonaceous deposit into a gaseous product. More particularly, the mathematical simulation can be used to determine the relationship between operating conditions and production of product for a given subterranean deposit to thereby permit prediction of the effect of a change of operating conditions on the product produced. In this manner the bioconversion conditions may be selected to provide a predicted result.

Well bores are defined as specific points or nodes located at a specific grid block location such as in FIG. 1. Well bores include injection wells IW, monitoring well bores MW and production well bores PW. The IW well is located in grid G8, production wells PW are located at the intersections 10 of the grid lines, such as lines 6′ and 6″. Other well bores are the monitoring wells MW whose locations are selected to monitor the predicted process and for use during implementation by the selection of an optimum predicted process. It should be understood that the construction of such wells is well known for both above surface structures and subsurface structures and need not be described herein. The well surface and subsurface constructions are schematically represented in the figures by the wells IW, MW and PW structures.

The above equations 1-8 and the corresponding process of FIGS. 2a and 2b establish the physical conditions at each grid G1-n location, dimensions in the X, Y and Z directions and parameters of the deposit, which if coal, such as coal density, porosity, permeability, fluid properties and so on. The simulation of the prediction process proceeds when a condition is imposed over a given time step, steps B and C, FIG. 2a. The input of water and nutrients, for example, can be defined for a given well at a specific flow rate, over a small time step, for example. 0.1 days, or the output of water or drop in pressure, at a given production recovery well PW, over a specific time step or any combination thereof. The equations and process then calculate the effect of that input conditions on all of the grids and the resulting conditions at each grid and node for that time step. Once the calculations reach convergence where the corresponding parameters for all equations are the same within the determined tolerance (they are iterative) the process then executes the next incremented time step, step C, FIG. 2a, and so on.

The predicted processes outputs at each of the grids are compared for output to determine the location of the different production recovery well bores in the implemented process based on optimized flows at the selected grid or grids for the inputted different selected prediction amounts of microbes, water, water flow rate and other imputed elements are inputted at the IW bore. Once the optimum results are selected, the production recovery wells are then produced at the designated locations in the grid, and actual input materials based on this prediction (the corresponding input assumptions) are inputted into the injection well IW. The outputs are measured at the production recovery wells and monitored at the monitoring wells for compliance with the prediction.

If one or more of the wells are not performing satisfactory according to the prediction, then a new prediction is selected from different new predictions based on selected new different inputs and outputs and these are then monitored and compared to the predictions and estimates made at the different wells. In this way optimum performance is obtained at all of the wells that best match the desired output predictions of expected optimum values for a given deposit based on determined empirical valuations.

The outputs are monitored at all PW and the deposit parameters may be monitored at the MW for compliance with the predictions on a periodic basis. If any of the wells exhibit a reduction in output as compared to the prediction, then the prediction process may be restarted based on new input parameters. Various iterations of this process may be conducted until a further estimated optimum process is predicted and selected, and the implementation process selected according to the new estimate and predictions and so on. Also new monitoring and production wells may be established, if the current monitoring wells do not correlate with the production well outputs or the predictions.

The above simulation modeling methodology is known as the Finite Difference Method (FDM). Conventional finite difference simulation is underpinned by three physical concepts: conservation of mass, isothermal fluid phase behavior, and the Darcy approximation of fluid flow through porous media. Thermal simulators (most commonly used for heavy-oil applications) add conservation of energy to this list, allowing temperatures to change within the reservoir. Finite difference models come in both structured and more complicated unstructured grids, as well as a variety of different fluid formulations, including black oil and compositional. An important application of finite differences is in numerical analysis, especially in numerical ordinary differential equations and numerical partial differential equations, which aim at the numerical solution of ordinary and partial differential equations respectively. The idea is to replace the derivatives appearing in the differential equation by finite differences that approximate them. The resulting methods are called finite difference methods.

There are other types of simulation methods that may be used for developing a mathematical simulation to predict gaseous product production from bioconverting a subterranean carbonaceous deposit based on one or more properties of the deposit, operating conditions, the microbial consortia and predicted changes in the deposit that result from the bioconversion, such as Finite Element, Streamline and Boundary Element methods.

The Finite Element Method (FEM) (sometimes referred to as Finite Element Analysis) is a numerical technique for finding approximate solutions of partial differential equations as well as of integral equations. The solution approach is based either on eliminating the differential equation completely (steady state problems), or rendering the partial differential equation into an approximating system of ordinary differential equations, which are then solved using standard techniques such as Euler's method, Runge-Kutta, etc. In solving partial differential equations, the primary challenge is to create an equation that approximates the equation to be studied, but is numerically stable, meaning that errors in the input data and intermediate calculations do not accumulate and cause the resulting output to be meaningless.

The differences between FEM and FDM are:
The finite difference method is an approximation to the differential equation; the finite element method is an approximation to its solution.
The most attractive feature of the FEM is its ability to handle complex geometries (and boundaries) with relative ease. While FDM in its basic form is restricted to handle rectangular shapes and simple alterations thereof, the handling of geometries in FEM is theoretically straightforward.
The most attractive feature of finite differences is that it can be very easy to implement.

Generally, FEM is the method of choice in all types of analysis in structural mechanics (i.e. solving for deformation and stresses in solid bodies or dynamics of structures) while computational fluid dynamics (CFD) tends to use FDM or other methods (e.g., finite volume method). CFD problems usually require discretization of the problem into a large number of cells/grid points (millions and more), therefore cost of the solution favors simpler, lower order approximation within each cell. This is especially true for ‘external flow’ problems, like air flow around the car or airplane, or weather simulation in a large area.

Reservoir simulation using Streamlines is not a minor modification of current finite-difference approaches, but is a radical shift in methodology. The fundamental difference is in how fluid transport is modeled. In finite difference models fluid movement is between explicit grid blocks, whereas in the streamline method, fluids are moved along a streamline grid that may be dynamically changing at each time step, and is decoupled from the underlying grid on which the pressure solution is obtained. Decoupling transport from the underlying grid can improve computational speed, reduce numerical diffusion and reduce grid orientation effects.

The paths traced by movement of fluid particles subjected to a potential gradient (or pressure gradient) are called streamlines. A tangent drawn to a streamline at a certain point represents the total velocity vector at that point. The streamline simulation is a technique that predicts multi-fluid displacements along the streamlines generated from numerical solutions to the diffusivity equation. The technique decouples computation of saturation variation from the computation of pressure variation in time and space. Using a finite difference method, the initial steady state pressure field is computed based on spatial variations in mobility, and is updated in response to significant time-dependent changes in mobility. The flow velocity field is then computed from the pressure field, and streamlines are traced based on the underlying velocity field. Streamlines originate at the injectors and culminate at producers. Once the streamline paths are determined, displacement processes are computed along the streamlines using 1-D, analytical or numerical models.

The Boundary Element Method (BEM) is a numerical computational method of solving linear partial differential equations which have been formulated as integral equations (i.e. in boundary integral form). It can be applied in many areas of engineering and science including fluid mechanics, acoustics, electromagnetics, and fracture mechanics. (In electromagnetics, the more traditional term “method of moments” is often, though not always, synonymous with “boundary element method”.)

The integral equation may be regarded as an exact solution of the governing partial differential equation. The boundary element method attempts to use the given boundary conditions to fit boundary values into the integral equation, rather than values throughout the space defined by a partial differential equation. Once this is done, in the post-processing stage, the integral equation can then be used again to calculate numerically the solution directly at any desired point in the interior of the solution domain. The boundary element method is often more efficient than other methods, including finite elements, in terms of computational resources for problems where there is a small surface/volume ratio. Conceptually, it works by constructing a “mesh” over the modeled surface. However, for many problems boundary element methods are significantly less efficient than volume-discretisation methods (Finite element method, Finite difference method, Finite volume method). Boundary element formulations typically give rise to fully populated matrices. This means that the storage requirements and computational time will tend to grow according to the square of the problem size. By contrast, finite element matrices are typically banded (elements are only locally connected) and the storage requirements for the system matrices typically grow quite linearly with the problem size. Compression techniques (e.g. multipole expansions or adaptive cross approximation/hierarchical matrices) can be used to ameliorate these problems, though at the cost of added complexity and with a success-rate that depends heavily on the nature of the problem being solved and the geometry involved.

BEM is applicable to problems for which Green's functions can be calculated. These usually involve fields in linear homogeneous media. This places considerable restrictions on the range and generality of problems to which boundary elements can usefully be applied. Nonlinearities can be included in the formulation, although they will generally introduce volume integrals which then require the volume to be discretised before solution can be attempted, removing one of the most often cited advantages of BEM. A useful technique for treating the volume integral without discretising the volume is the dual-reciprocity method. The technique approximates part of the integrand using radial basis functions (local interpolating functions) and converts the volume integral into boundary integral after collocating at selected points distributed throughout the volume domain (including the boundary). In the dual-reciprocity BEM, although there is no need to discretize the volume into meshes, unknowns at chosen points inside the solution domain are involved in the linear algebraic equations approximating the problem being considered.

The Green's function elements connecting pairs of source and field patches defined by the mesh form a matrix, which is solved numerically. Unless the Green's function is well behaved, at least for pairs of patches near each other, the Green's function must be integrated over either or both the source patch and the field patch. The form of the method in which the integrals over the source and field patches are the same is called “Galerkin's method”. Galerkin's method is the obvious approach for problems which are symmetrical with respect to exchanging the source and field points. In frequency domain electromagnetics this is assured by electromagnetic reciprocity. The cost of computation involved in naive Galerkin implementations is typically quite severe. One must loop over elements twice (so we get n² passes through) and for each pair of elements we loop through Gauss points in the elements producing a multiplicative factor proportional to the number of Gauss-points squared. Also, the function evaluations required are typically quite expensive, involving trigonometric/hyperbolic function calls. Nonetheless, the principal source of the computational cost is this double-loop over elements producing a fully populated matrix.

The Green's functions, or fundamental solutions, are often problematic to integrate as they are based on a solution of the system equations subject to a singularity load (e.g. the electrical field arising from a point charge). Integrating such singular fields is not easy. For simple element geometries (e.g. planar triangles) analytical integration can be used. For more general elements, it is possible to design purely numerical schemes that adapt to the singularity, but at great computational cost. Of course, when source point and target element (where the integration is done) are far-apart, the local gradient surrounding the point need not be quantified exactly and it becomes possible to integrate easily due to the smooth decay of the fundamental solution. It is this feature that is typically employed in schemes designed to accelerate boundary element problem calculations.

The predicted processes outputs at each of the grids are compared for output to determine the location of the different production recovery well bores in the implemented process based on optimized flows at the selected grid or grids for the inputted different selected prediction amounts of microbes, water, water flow rate and other imputed elements are inputted at the IW bore. Once the optimum results are selected, the production recovery wells are then produced at the designated locations in the grid, and actual input materials based on this prediction (the corresponding input assumptions) are inputted into the injection well IW. The outputs are measured at the production recovery wells and monitored at the monitoring wells for compliance with the prediction.

The mathematical model as described herein enables the understanding and prediction of the response of the subterranean formation to a range of inputs, such as the injection of fluids or gases into the subterranean formation and the production of fluids and gases from the subterranean formation. With a further understanding of the physical properties of the subterranean formation, such as the Young's Modulus of Elasticity, and rock compressibility, and the relationship of the formation characteristics with regard to its porosity and permeability, the mathematical model may be employed to predict how the injection and withdrawal of fluids and/or gases may affect pressure, permeability, porosity and fluid movement within, throughout and at various locations across the subterranean formation.

Further, with an understanding of how microbes may be introduced, how the microbes may grow, how the microbes may be carried with fluids and gases flowing within the subterranean formation, how they may attach themselves to the surfaces of the subterranean formation, how they may grow in population by cell division, how they may be reduced in population by cell death, how they may utilize introduced nutrients, how the nutrients may be introduced, how the nutrients may move throughout the subterranean formation, how the nutrients may be consumed by the microbes, how the metabolic products of the nutrients such as volatile fatty acids, acetate, methane and carbon dioxide may be produced, how these metabolic products may be adsorbed or desorbed within the subterranean formation, how the metabolic products may flow within the subterranean formation, how the metabolic products may be produced from the subterranean formation to the surface, the model may be employed to predict how microbes may be utilized for the generation and production of methane, carbon dioxide and other hydrocarbons from said formation.

In addition, with an understanding of the constituents, spatial distribution and other characteristics of the subterranean formation, and an understanding of how microbes may interact with the subterranean formation in the biological conversion of said formation carbon-bearing matter to methane, carbon dioxide and other hydrocarbon products, the mathematical model may be utilized to predict how said subterranean formation may be changed vertically and areally in terms of volume, porosity, permeability, and composition under a range of conditions. As bioconversion of the carbon-bearing subterranean formation proceeds, solid matter is converted to gases and liquids, such as methane, carbon dioxide, and volatile fatty acids, as well as other hydrocarbons and solids fines. This reduction in the solid volume of the carbon-bearing subterranean formation may substantially change the composition of the remaining solid, as well as the porosity and permeability of the subterranean formation, its spatial distribution of porosity and permeability, and the volume of fluids, microbes, and nutrients and their flow, distribution and concentration within said subterranean formation. Further, these various characteristics of the formation and the fluids, gases, microbes and nutrients therein may vary with changes in pressure, temperature, saturation and flow as a function of time.

The calculation model of the invention may be utilized to predict the flow rates of methane-(or other gases such as carbon dioxide and other hydrocarbons) from the subterranean formation under a wide range of conditions. The calculation model may also be utilized to predict the amount or volume of the subterranean formation that may be biologically converted to methane (or carbon dioxide and other hydrocarbons), and the location and extent of such conversion, under a range of conditions and as a function of time.

The calculation model of the invention may also be utilized in a continuous or near-continuous or periodic fashion to assess the efficiency of an in-situ biological conversion process, to predict how the process may be affected by changes in input or operating conditions, changes in nutrient inputs, changes in pressure, changes in nutrients application, and changes in formation composition and water geochemistry.

The model of the invention may also be utilized to predict the rates of production of methane, carbon dioxide and other hydrocarbons from the subterranean formation as a function of time and at various points across and within the subterranean formation that is affected by the biological conversion process.

The model may also be utilized to predict how the rates of production of methane, carbon dioxide and other hydrocarbons may be affected under a variety of input conditions, such as the location, spacing, and orientation of wellbores drilled into said subterranean formation, and the rates, timing, duration and location of inputs of fluids, gases, chemicals used to treat the deposit, methanogenic consortia and nutrients through such wellbores, and the rates, timing, duration, and location of production of fluids, gases and nutrients from such wellbores.

The model may also be utilized to predict how the movement of fluids, microbes, nutrients, methane, carbon dioxide and other hydrocarbons may be affected by changes in the subterranean formation permeability, porosity, volume and characteristics.

The model may also be utilized to predict the extent and location of subterranean formation bioconversion under variable conditions of the flow of fluids, microbes, nutrients, methane, carbon dioxide and other hydrocarbons, the pressure of the formation, areally and over time.

The model may be utilized to optimize the rate, extent and efficiency of the bioconversion of the carbon-bearing subterranean formation to methane, carbon dioxide and other hydrocarbons under a variety of conditions and by making adjustments to such conditions over time, measuring the results, utilizing the model to match the results to operating conditions and making further adjustments to operating conditions, in a continuous, near-continuous or periodic fashion.

The model may be utilized to predict how chemicals such as surfactants, solubilization agents, pH buffers, oxygen donor chemicals and bio-enhancing agents may be introduced into, flow through, be adsorbed and/or desorbed, be produced from, and change the volume, permeability and porosity characteristics of the subterranean formation; how such chemicals may affect the growth, population, movement, death of microbes in the subterranean formation, and how such chemicals may affect the generation, flow, adsorption, desorption and production of methane, carbon dioxide and other hydrocarbons from the subterranean formation.

The model may be used to predict how gases such as hydrogen, carbon dioxide and carbon monoxide may be introduced into, flow through, be adsorbed and/or desorbed, be produced from, and change the volume, permeability and porosity characteristics of the subterranean formation; how such gases may affect the growth, population, movement, death of microbes in the subterranean formation, and how such gases may affect the generation, flow, adsorption, desorption and production of methane, carbon dioxide and other hydrocarbons from the subterranean formation.

The model may be utilized to predict how electrical current may be applied to affect the growth, population, movement and death of microbes in the subterranean formation, and the generation, flow, adsorption, desorption and production of methane, carbon dioxide and other hydrocarbons from the subterranean formation.

The model may be utilized to design systems, including the placement of wellbores; the design of facilities, including flow lines, vessels, pumps, compressors, mixers, and tanks; and the operation of wellbores and facilities in order to optimize the bioconversion of carbon and other materials in the subterranean formation to methane, carbon dioxide and other hydrocarbons, and the production and recovery of methane, carbon dioxide and other hydrocarbons from said subterranean formation.

The model may be integrated with a mathematical probability and/or statistical analysis model in order to enable stochastic assessment of a range of variables and conditions of the model, and to provide a range of possible outcomes resulting from a range of input and/or operating conditions applied.

The model may further be integrated with an economics or financial analysis model to assess the economic viability of implementation of a process or processes for the conversion of carbon and other materials contained in the subterranean formation to methane, carbon dioxide and other hydrocarbons under a range of input and operating conditions, system designs and capital and operating costs assumptions.

The model may further be integrated with both a mathematical probability and/or statistical analysis model and an economics or financial analysis model to assess the economic viability of implementation of a process or processes for the conversion of carbon and other materials contained in the subterranean formation to methane, carbon dioxide and other hydrocarbons under a range of input and operating conditions, system designs and capital and operating costs, and with any number of risk and/or probability distributions of inputs to said model. In this embodiment, the fully integrated mathematical model, probability model and financial analysis model will enable the evaluation of a comprehensive range of possible systems designs, operating conditions, variable conditions, geological and geophysical conditions and inputs and the assessment of economic potential of the processes under consideration.

The calculation model may be utilized in conjunction with mathematical probability and/or statistical analysis models to enable stochastic assessment of a range of variables and conditions and to provide a range of possible outcomes resulting from a range of input and/or operating conditions that are applied. This utilization may be achieved by one of ordinary skill in the mathematical art.

The model may also be incorporated with or integrated with an economics or financial analysis model to assess the economic viability of implementation of a process(s) for the conversion of hydrocarbon or other materials contained in the subterranean formation to methane, carbon dioxide and other hydrocarbons under a range of input and operating conditions, system designs, and capital and operating cost assumptions any number of risk and/or probability distributions of inputs to said model.

The calculation model may be utilized to assess the extent and location of the bioconversion materials in the subterranean deposit formation to methane, carbon dioxide or other hydrocarbons.

The model of the invention may be utilized to manipulate, adjust, change or alter and control the systems of the bioconversion process via comparing actual operational results and the data to model-predicted results.

The volumes and mass of the deposit, porosity, fluid, gas(s), nutrients, and biological materials may be determined or estimated at any given time before, during and after the bioconversion process is implemented.

The overall efficiency of the calculation model for the bioconversion of the hydrocarbon deposit may be determined or estimated during or after the model process is applied.

It should be understood that the embodiments described herein are given by way of illustration and not limitation and that one of ordinary skill may make modifications to the disclosed embodiments. For example, while one injection well is described, there may be any number of such wells and corresponding production wells in a given implementation and according to a given hydrocarbon formation. It is intended that the scope of the invention be determined in accordance with the appended claims.

What is claimed is:

1. A method of employing a comprehensive mathematical model that fully describes the geological, geophysical, hydrodynamic, microbiological, chemical, biochemical, geochemical, thermodynamic and operational characteristics of systems and processes for in-situ bioconversion of carbon-bearing subterranean formations to methane, carbon dioxide and other hydrocarbons using indigenous or non-indigenous methanogenic consortia, via the introduction of microbial nutrients, methanogenic consortia, chemicals and electrical energy, and the operation of the systems and processes via surface and subsurface facilities.

2. A method for the design, implementation and optimization of systems and processes for the in-situ bioconversion of carbon-bearing subterranean formations to methane, carbon dioxide and other hydrocarbons using indigenous or non-indigenous methanogenic consortia via the introduction of microbial nutrients, methanogenic consortia, chemicals and electrical energy, utilizing a comprehensive mathematical model that fully describes the geological, geophysical, hydrodynamic, microbiological, chemical, biochemical, geochemical, thermodynamic and operational characteristics of such systems and processes.

3. The method according to claim 2 including utilizing the model for assessing the extent and location of the bioconversion of materials in the subterranean deposit formation to methane, carbon dioxide and/or other hydrocarbons.

4. The method according to claim 2 including manipulating, adjusting, changing or altering and controlling the bioconversion of materials in the subterranean formation to methane, carbon dioxide and of the bioconversion process via comparing actual operational results and the data to model-predicted results.

5. The method according to claim 2 including determining or estimating the volumes and mass of subterranean formation, porosity, fluid, gas, nutrient and biological material at any given time before, during and after applying the method of claim 2.

6. The method according to claim 2 including determining the amount of carbon in the subterranean formation that is bioconverted to methane, carbon dioxide and other hydrocarbons, at any given time before, during and after applying the method according to claim 2.

7. The method of claim 2 including utilizing any of a variety of solution methods including at least one of finite difference, finite element, streamline and boundary element for the mathematical model.

8. A process for producing a gaseous product by bioconversion of a subterranean carbonaceous deposit, comprising:
bioconverting a subterranean carbonaceous deposit to the gaseous product by use of a methanogenic consortia, said bioconverting being operated based on a mathematical simulation that predicts production of the gaseous product by use of at least (i) one more physical properties of the deposit; (ii) one or more changes in one or more physical properties of the deposit as result of said bioconverting; (iii) one or more operating conditions of the process; and (iv) one or more properties of the methanogenic consortia.

9. The process of claim 8 wherein the one or more physical properties of the deposit comprise depth, thickness, pressure, temperature, porosity, permeability, density, composition, types of fluids and volumes present, hardness, compressibility, nutrients, presence, amount and type of methanogenic consortia.

10. The process of claim 8 where the operating conditions comprise injecting into the deposit: a predetermined amount of the methogenic consortia, a predetermined amount of water at a predetermined flow rate, and a predetermined amount of a given nutrient, wherein the temperature of all of the foregoing predetermined.

11. The process of claim 8 wherein the properties of the methanogenic consortia include the types and amount of consortia.

12. The process of claim 8 wherein the gaseous product is one of methane and carbon dioxide.

13. The process of claim 8 wherein the gaseous product is at least one gas, the process including recovering the at least one gas from the deposit.

14. The process of claim 8 wherein the process includes recovering the at least one gas from the deposit and the simulation includes dividing the deposit in to at least one grid of a plurality of three dimensional deposit subunits, and predicting the amount of recovery of the at least one gas from each subunit.

15. The process of claim 8 wherein the simulation includes dividing the deposit into a grid of a plurality of three dimensional subunits, selecting the subunit exhibiting an optimum amount of gaseous product to be recovered and then recovering the bioconverted product from that selected subunit.

16. The process of claim 8 including recovering the gaseous product from the deposit wherein the simulation includes dividing the deposit in to at least one grid of a plurality of three dimensional deposit sectors, and predicting the amount of recovery of the at least one gas from each sector, and determining the flow of the gaseous product from sector to adjacent sector.

17. The process of claim 8 wherein the simulation comprises the steps of FIGS. 2a and 2b.

18. The process of claim 8 wherein the simulation comprises the simultaneous solution of equations 1-12.

19. The process of claim 8 wherein the simulation comprises solving equations 1-12 for each unknown parameter in these equations until the value of that parameter reaches a corresponding range within a given tolerance for that parameter over a time step period.

20. The process of claim 19 wherein the simulation comprises repeating the solution of the equations for different time step periods until the value of each parameter reaches said range.
